(19) United States
(12) Patent Application Publication
(10) Pub. No.: US 2016/0262620 A1
(43) Pub. Date: Sep. 15, 2016

(54) WIRELESS LEADWIRE PLUG

(71) Applicant: Börje Rantala, Helsinki (FI)

(72) Inventor: Börje Rantala, Helsinki (FI)

(21) Appl. No.: 14/658,142

(22) Filed: Mar. 14, 2015

(51) Int. Cl. A61B 5/00; A61B 5/0428; A61B 5/1455; A61B 5/0404; A61B 5/0205; A61B 5/04; A61B 5/0408

(52) U.S. Cl. CPC A61B 5/0006; A61B 5/04012; A61B 5/04288; A61B 5/0408; A61B 5/0024; A61B 5/0404; A61B 5/6898; A61B 5/0205; A61B 5/14552; A61B 2560/0209; A61B 2560/0214; A61B 5/0809

(57) ABSTRACT

The present invention is a novel approach in combining wired and wireless ECG monitoring. An ECG Wireless Plug (“Wplug”) is inserted between the Trunk Cable and the Leadwire Cable, so as to both pass the ECG to the Host Monitor and to intercept the signal for processing and wireless transmission.

When the Wplug is inserted and the Host Monitor is active, the signal transmitted to the Telemetry Receiver can be identified as belonging to the same patient as on the Host Monitor.

### BACKGROUND OF THE INVENTION

This disclosure relates generally to patient monitoring. Particularly to ECG monitoring.

When a patient is being monitored by ECG it is greatly desirable to do it wirelessly, as nurses have problems with tangled cables, and patients need to be mobile.

Multiple ways for managing ECG and telemetry cabling are available and have been proposed as described in [1]. Telemetry also has problems, e.g. battery life, battery change, battery charging, pairing of the patient worn transmitter with the receiver/display after each disconnect (typical after battery change or charging). Pairing is a specific problem in wireless monitoring, as it has to be clear which patient connects to which ECG waveform (and other display, alarms etc.); a problem not encountered with wired monitoring. Other issues with telemetric monitoring is its limited compatibility with wired monitoring, especially with patient occasionally mobile, e.g. toilet visits. The switching between devices is cumbersome and error prone.

### BRIEF DESCRIPTION OF THE INVENTION

The present invention is a novel approach in combining wired and wireless ECG monitoring. Here an ECG Wireless Plug (“Wplug”) is inserted between the Trunk Cable and the Leadwire Cable, so as to both pass the ECG to the Host Monitor, and to intercept the signal for processing and wireless transmission.

One preferred embodiment of this is a Wplug that has two connectors on opposite sides, one Trunk connector intended to mate with the Leadwires and one Leadwire connector intended to mate with the Trunk Cable. The Wplug has its own battery to feed the electronic part that intercepts the ECG on its way to the Host Monitor, processes it and sends it wirelessly to a Wireless Host, e.g. a Smartphone, a Central Station or the Host Monitor wireless receiver. This “Basic” embodiment has the advantage of not requiring modifications to the Host Monitor, but has limited functionality and must have its own battery replacement or recharging arrangements.

Another preferred embodiment is a version can charge the battery from the Host Monitor, but requires modification of either the Host Monitor or the Trunk Cable.

Still another preferred embodiment uses digital communication, e.g. USB, with the host.

Both the charging and the USB communication can be embedded in the redundant shield pins in a typical Leadwire and Trunk Cable connectors.

All versions can also support identifying the patient on the Wireless device (Pairing) by, at least briefly, have both the Host Monitor and the Wplug capture the ECG. The Wireless Host then compares the, initially unpaired, ECG waveform stream from the Wplug with a set of candidate ECG waveforms streams of Named Patients (normally the patients of the supported hospital department). Correlating the waveforms or physiological parameters derived from the waveforms (R-R intervals, Heart Rate . . . ) produces a Pairing with virtually 100% reliability.

Still another preferred embodiment uses the Wplug Host Monitor side connector (the “L” connector) to allow connection to an expanded set of Leadwires C2 . . . C6, also known as the Chest Leads or Precordial Leads in a 12 Lead ECG. As implemented in FIG. 3, this disables the Host Monitor connection by occupying the “L” connector, but it is straightforward to implement more complex connector adapters or Wplugs, where this is still possible. Of course, in the simple version, the pairing must be done in 5 Lead mode.

### DESCRIPTION OF THE DRAWINGS

FIG. 1 illustrates the Basic embodiment the invention; here the Wplug in shown inserted between the Trunk Cable and the Leadwire Cable. The Wplug can communicate directly to a Telemetry Receiver, which can be a computer behind a Wi-Fi access point, a Host monitor, or a Smartphone close to the patient.

FIG. 2 shows a Basic Wplug design for wide compatibility with legacy Host Monitors. The Wplug has connectors that match the Trunk Cable and the Leadwire Cable. It passes the ECG signals through and captures them for its own processing.

FIG. 3 shows an extended capability design with battery charging and possible wired digital communication.

FIG. 4 shows a possible use of the Host Monitor side of the Wplug when in non-Host connected mode, where the connector is unused. A version of the Wplug can support full 12 Lead ECG by utilizing the Host port connector as a Chest Lead connector.

### DETAILED DESCRIPTION OF THE INVENTION

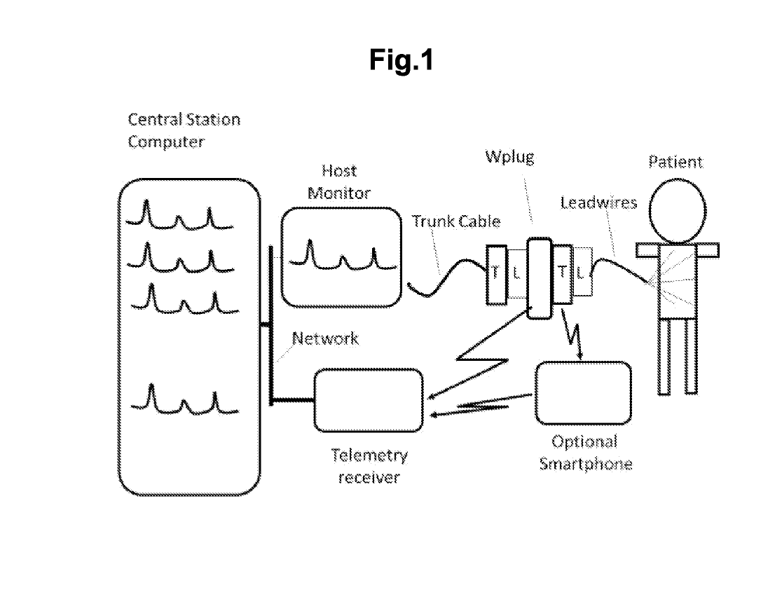

FIG. 1 illustrates the Basic embodiment the invention; here the Wplug in shown inserted between the Trunk Cable and the Leadwire Cable. The Wplug can communicate directly to a Telemetry Receiver, which can be a computer behind a Wi-Fi access point, a Host monitor, or a Smartphone close to the patient. When communicating locally within a short distance from the Host Monitor, the preferred communication could be Bluetooth Low Energy (BLE) or Body Area Network (BAN, [4]). For extended patient mobility, the preferred communication is Wi-Fi or traditional telemetry channels (e.g. WTMS in the US). As it is desirable to have a display at the patient side, a patient dedicated Smartphone is preferred; the Wplug communicates BLE to the Smartphone, which processes the ECG and displays and alarms locally in addition to transmitting to the Telemetry Receiver over the hospital Wi-Fi network.

Having the processing close to the patient is advantageous, as the effects of communication disturbances are minimized.

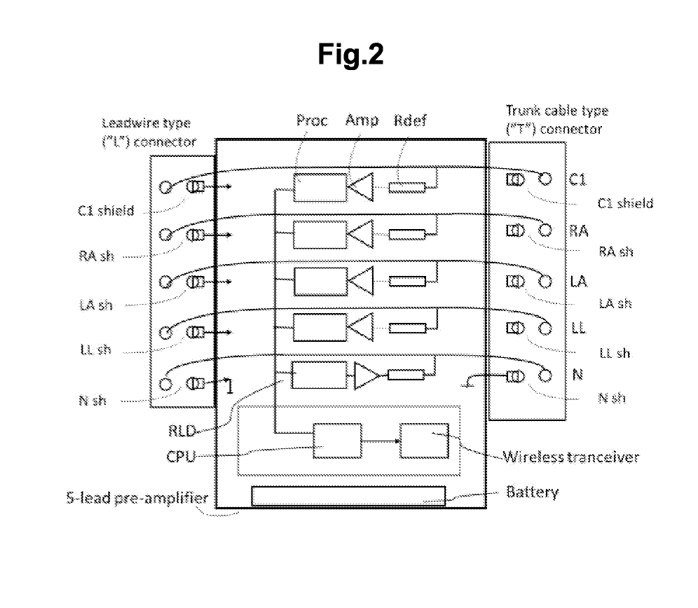

FIG. 2 shows a Basic Wplug design for wide compatibility with legacy Host Monitors. The Wplug has connectors that match the Trunk Cable and the Leadwire Cable. It passes the ECG signals through and captures them for its own processing.

The detailed design of “eavesdropping” on the signals from the patient to the host is crucial. The Wplug must show high impedance in all eavesdropping connections so as not to reduce ECG performance of the Host Monitor. Note that the Host Monitor typically has defibrillation protection resistors in its front end, which means that during defibrillation the voltage between any leads can rise to 5000 volts. Also, the Host Monitor may use Impedance Respiration, meaning it feeds 30-100 kHz current to the patient and measures chest impedance variations. None of this can be disturbed by the Wplug.

Special attention must be given to the Right Leg Drive (RLD), a standard way of reducing noise in an ECG monitor, as it involves active feedback from the ECG amplifier system into the patient. The Wplug RLD must be very weak or switchable to off when the Host is connected. Host connection can be detected by the Wplug in various ways: by using the standard Leads Off mechanism, where a small current is injected into the electrodes to measure DC or AC impedance, or by detecting the presence of the shield connections (Rash, Lash . . . ) which the host connects to floating ground. Fortunately the need for RLD in wireless mode is much smaller than in standard Host Monitor mode, as the leakage current through the Host Monitor ECG floating front end is essentially missing. The preferred design is weak RLD by using a large value for the defibrillation resistor Rdef.

Also the Leads Off mechanism should be flexible and adapt to the Host Monitor Leads Off mechanism when connected. Otherwise the mechanisms will interfere.

The Basic design has a replaceable battery (alkaline, lithium, rechargeable) or a built in battery. In the case of a replaceable battery, it is desirable to have a super capacitor as internal backup, as it enables operation during the time the battery is removed from the Wplug. With good industrial design battery replacement takes less than one minute, which a 1 Farad super capacitor can easily support.

If the Wplug is replaced with a second device while the first one is removed for charging, the Pairing needs to be re-performed. The Wireless Receiver detects the break in operation, and automatically performs or requests a re-Pairing.

Small variations in the connectors are included in the invention. Examples of these are the use of 6 Lead ECG connectors instead of 5 Leads (6 Leads adds C2 to the standards 5 Lead connector. Also a unified 3-5-6-12 Lead connector can be used [2].

A 3 Lead cable can be used on the patient side, but the Host port must use at least a 5 Lead connector that has a dedicated N pin.

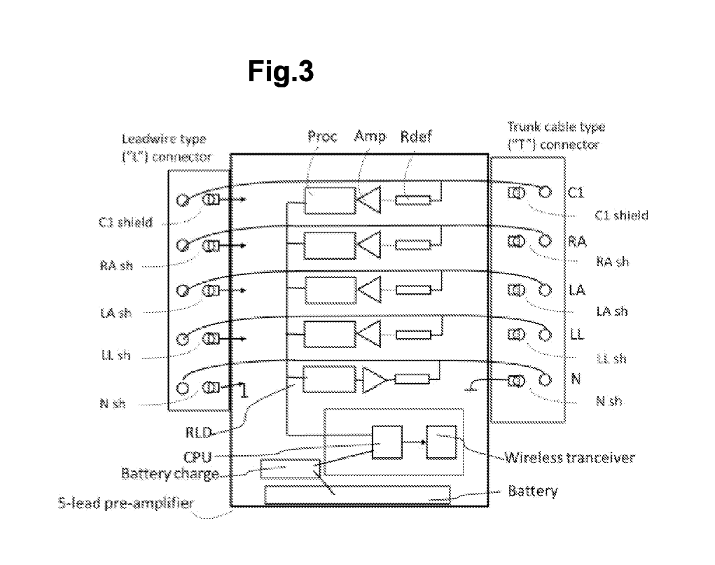

FIG. 3 shows an extended capability design with battery charging and possible wired digital communication. The extended design can be achieved in several ways. The simplest way of enabling Wplug battery charging or battery saving when Host connected, is to use a dedicated Trunk Cable with two connectors at the Host Monitor end. One goes to a legacy Host Monitor and the other forks to a battery or floating power supply that is used to feed the Wplug battery charging power.

It is obviously much more elegant to have Host Monitor support for power. In case of a Host Monitor that is supportive of the Wplug, it is useful to add a direct wired communication modality to the Wplug, e.g. USB. This can be used for several purposes, e.g. making the pairing faster and less dependent on correlating ECG waveforms. Again, the easiest way of doing this is using the redundant “T” and “L” connector shield ground pins.

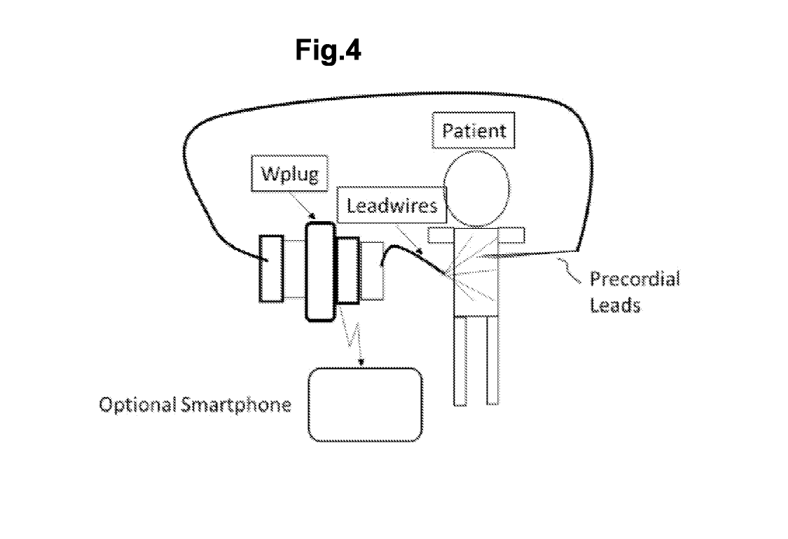

FIG. 4 shows a possible use of the Host Monitor side of the Wplug when in non-Host connected mode, where the connector is unused. A version of the Wplug can support full 12 Lead ECG by utilizing the Host port connector as a Chest Lead connector. It is also possible to allow usage of standard 5+5 pin Leadwire sets by adding a “T”-“T” adapter on the Host port.

Normally this allows 12 L ECG only when in non-Host mode, but it is obvious that a multiport adapter is easy to design, which would allow all modalities to be used simultaneously.

The Wplug can also be used for other physiological monitoring, e.g. a pulse oximeter. In addition to dedicated connector for this, a Host port that is redundant when not connected to a Host Monitor, can be used to connect an oximeter or an oximeter probe.

### REFERENCES CITED

### US Patents

[1] ECG lead system
U.S. Pat. No. 8,897,865 B2 Justin Farrior, 21 Oct. 2009
Original Assignee Covidien Lp
[2] System for ECG monitoring with selective lead switching
U.S. Pat. No. 6,553,250 B2 Börje Rantala, 8 Jul. 1999
Original Assignee Instrumentarium Corp.
[3] Wireless ECG system
U.S. Pat. No. 7,403,808 B2 Rud Istvan et. al. 17 Jul. 2001
Original Assignee Lifesync Corporation

### Other Publications

4] Sana Ullah, Henry Higgins, Bart Braem, Benoit Latre, Chris Blondia, Ingrid Moerman, Shahnaz Saleem, Ziaur Rahman and Kyung Sup Kwak, A Comprehensive Survey of Wireless Body Area Networks: On PHY, MAC, and Network Layers Solutions, Journal of Medical Systems (Springer), 2010. doi:10.1007/s10916-010-9571-3.

1. A device (Wplug) for insertion between a Host Monitor and the patient ECG Leadwires that captures the ECG signals for processing and wireless transmission.

2. The device according to claim 1 that allows the Host Monitor to function essentially undisturbed.

3. The device according to claim 1 where the Wplug functions as a telemetry monitor when the connection to the Host Monitor is broken.

4. The device according to claim 1, that uses two 5 Lead connectors, one that connects to the Leadwires and one that connects to the ECG Trunk Cable.

5. The device according to claim 1, that uses two 6 Lead connectors, one that connects to the Leadwires and one that connects to the ECG Trunk Cable.

6. The device according to claim 1, where the simultaneous acquisition of two ECG waveform streams is used to identify the patient on the wireless stream (Pairing).

7. The device according to claim 6, where the Pairing is performed by correlating physiological parameters derived from the ECG waveforms from the Host Monitor and from the Wplug.

8. The device according to claim 1, where the Host side connector is used as a connector for additional chest electrodes.

9. The device according to claim 1, where the battery is charged when power availability is detected.

10. The device according to claim 1, where the wireless connection is Bluetooth Low Energy to a patient dedicated Smartphone.

11. The device according to claim 1, where Host side connector is used as a connector for additional patient worn sensors.

12. The device according to claim 1, where a digital communication channel uses redundant shield connector pins of the Host side connector.

13. The device according to claim 1, where the device supports additional physiological measurements.

14. The device according to claim 13, where the additional physiological measurement is a pulse oximeter.

15. The device according to claim 14, where the pulse oximeter probe uses redundant shield connector pins.

16. The device according to claim 1, where the Right Leg Drive is weak compared to Host Monitor.

17. The device according to claim 1, where Leads off is detected passively when connected to the Host Monitor.

18. The device according to claim 1, where a super capacitor is used for continued operation while the battery is being replaced.

19. The device according to claim 13, where the additional physiological measurement is impedance respiration or impedance cardiac monitoring.

20. The device according to claim 9, where the charger is a separate device not connected to a Host Monitor.
